(19) United States
(12) Patent Application Publication
(10) Pub. No.: US 2024/0237929 A1
(43) Pub. Date: Jul. 18, 2024

(54) POINT-OF-USE CALIBRATION SYSTEM FOR IRON ION DETECTION SYSTEM

(71) Applicant: Acies Medical LLC, Saint Paul, MN (US)

(72) Inventor: Brian L. Norling, Santa Barbara, CA (US)

(73) Assignee: Acies Medical LLC, Saint Paul, MN (US)

(21) Appl. No.: 18/559,203

(22) PCT Filed: May 18, 2022
(86) PCT No.: PCT/US2022/029827

§ 371 Date: Nov. 6, 2023

Related U.S. Application Data
(60) Provisional application No. 63/190,250, filed on May 19, 2021.

(51) Int. Cl. A61B 5/1495; A61B 5/00; A61B 5/145

(52) U.S. Cl. CPC A61B 5/1495; A61B 5/14503; A61B 5/14546; A61B 5/742; A61B 2560/0223

(57) ABSTRACT

A point-of-care ion calibration system that may utilize acquis solutions that contain different concentrations of ions. In some instances, the system utilizes a multi-chambered device wherein each separate chamber may contain a different concentration of ions. These concentrations may calibrate an ion detector which may be disposed within the end of a needle. The ion detector may pass into each chamber to obtain an ion level that may be feedback to an end-user. If the end-user cannot differentiate the feedback from varying ion concentrations, then the detector may not be working correctly and may be discarded.

### FIELD OF THE DISCLOSURE

The present disclosure relates to systems and methods for detecting biological substances. More particularly, it relates to systems and methods for detecting biological substances in conjunction with medical devices for the purposes of calibration of the medical device and for the assurance to an end-user that the medical device is functioning correctly prior to its interaction with a patient.

### BACKGROUND

Efforts to improve surgical outcomes and cost structure, particularly with spinal surgery, have led to increased use of minimally invasive procedures. These procedures often use image-guided modalities such as fluoroscopy, CT, nerve stimulators, and, more recently, the Doppler ultrasound test. However, while often involving less risk than surgery, minimally invasive spinal procedures, pain management procedures, nerve blocks, ultrasound-guided interventions, biopsy, and percutaneous placement or open intra-operative placement continue to carry risks of ineffective outcome and iatrogenic injuries, such as infection, stroke, paralysis and death due to penetration of various structures including, but not limited to, organs, soft tissues, vascular structures, and neural tissue such as, catastrophically, the spinal cord. In addition, injuries can occur regardless of practitioner experience because a surgical instrument must proceed through several layers of bodily tissues and fluids to reach the desired space in the spinal canal.

To illustrate, the intrathecal (or subarachnoid) space of the spinal region, where many medications are administered, houses nerve roots and cerebrospinal fluid (CSF) and lays between two of the three membranes that envelope the central nervous system. The outermost membrane of the central nervous system is the dura mater, the second is the arachnoid mater, and the third, and innermost membrane, is the pia mater. The intrathecal space is in between the arachnoid mater and the pia mater. A surgical instrument may need to first breach skin layers, fat layers, the interspinal ligament, the ligamentum flavum, the epidural space, the dura mater, and the subdural space to get to the intrathecal space. Additionally, in the case of a needle used to administer medication, the entire lumen opening must be within the sub-arachnoid space.

Because of the complexities involved in inserting a surgical instrument into the intrathecal space, penetration of the spinal cord and neural tissue are known complications of minimally invasive spine procedures and spine surgery. Additionally, some procedures require the use of larger surgical instruments. For example, spinal cord stimulation, a form of minimally invasive spinal procedure wherein small wire leads are inserted in the spinal epidural space, may require that a 14-gauge needle be introduced into the epidural space to thread the stimulator lead. Needles of this gauge are technically more difficult to control, posing a higher risk of morbidity. Complications can include dural tear, spinal fluid leak, epidural vein rupture with subsequent hematoma, and direct penetration of the spinal cord or nerves with resultant paralysis. These and other high-risk situations, such as spinal interventions and radiofrequency ablation, can occur when a practitioner is unable to detect the placement of the needle or surgical apparatus tip in critical anatomic structures.

At present, detection of such structures is operator-dependent, wherein operators utilize tactile feel, contrast agents, anatomical landmark palpation, and visualization under image-guided modalities. The safety of patients can rely upon the training and experience of the practitioner in tactile feel and interpretation of the imagery. Even though additional training and experience may help a practitioner, iatrogenic injury can occur independently of practitioner experience and skill because of anatomic variability, which can arise naturally or from repeat procedures in the form of scar tissue. Fellowship training in some procedures, such as radiofrequency ablation, may not be sufficiently rigorous to ensure competence; even with training, outcomes from the procedure can vary considerably. In the case of epidural injections and spinal surgery, variability in the thickness of the ligamentum flavum, width of the epidural space, dural ectasia, epidural lipomatosis, dural septum, and scar tissue all can add challenges to traditional verification methods even for highly experienced operators. Additionally, repeat radiofrequency procedures done when nerves regenerate, often a year or more later, are often less effective and more difficult because the nerves' distribution after regeneration creates additional anatomic variability.

No device exists that provides objective, reliable, consistent, real-time feedback of critical tissues and bodily fluids. Further, even the concept of objective device feedback has not been accepted by proceduralists, even though millions of spinal procedures are performed annually as a standard of care throughout the world.

### SUMMARY OF THE INVENTION

Provided is a calibration verification system for an iron ion detection needle. The disclosed verification system presents a simple method for verifying the accuracy and capability of a blood detection needle at a point of use. In one such embodiment, a point-of-use calibration system for an ion detection system may include at least a first chamber and a second chamber, wherein the first chamber may include a first ionic solution, and the second chamber may include a second ionic solution. The point-of-use calibration system may also include an upper opening and a lower opening being a part of the first chamber and a single opening in the second chambered. A seal may then be disposed between the lower opening of the first chamber and the opening of the second chamber. A cap can be disposed on the upper opening. A common axis can exist between the first chamber, the second chamber, the seal, and the cap.

A further embodiment may include a first ionic solution that contains a first concentration of iron ions, and a second ionic solution that contains a second concentration of iron ions. The two concentrations can have distinct differences in their concentration. The seal and cap may consist of a needle-pierceable material. This embodiment may also include an ion-detection needle having a proximal end and a distal end, wherein an ion detector is disposed on the distal end. This may also include a syringe connected to the ion-detection needle's proximal end and where the ion detector is in communication with a feedback system. Such feedback systems may include an indicator light, a numeric digital display, a variable tone generator, a variable color display, and combinations thereof.

An additional embodiment of a multi-chambered vial may include at least two chambers wherein the at least two chambers are aligned on a common vertical axis. A first chamber may be a top chamber, and a second chamber may be a bottom chamber when aligned vertically along the axis. The top chamber may have an upper opening and a lower opening, and the bottom chamber may have an opening. A pierceable cap can be disposed on the top opening of the top chamber, while a pierceable seal may be disposed between every chamber of the at least two chambers. Within the top and bottom, there be at least two acquis solutions wherein the at least two acquis solutions consist of different ionic concentrations, each chamber having a different acquis solution.

In some embodiments, there may be a retaining ring that can be disposed on the pierceable cap and around each pierceable seal. Other embodiments may include other nested chambers between the top and bottom chambers; for example, wherein the at least two chambers may include a third chamber having an upper opening and a lower opening, wherein the third chamber may be disposed between the top chamber and the bottom chamber, and wherein the third chamber may have a different ionic concentration than the first and second chambers.

One potential method for using a point-of-use calibration system as an ion detection system may include the steps of providing a multi-chambered vial comprising: at least two chambers wherein the at least two chambers are aligned on a common vertical axis; a first chamber of the at least two chambers is a top chamber and a second chamber of the at least two chambers is a bottom chamber, wherein the top chamber has an upper opening and a lower opening and the lower chamber has an opening; a pierceable cap disposed on a top opening of the top chamber; a pierceable seal disposed between every chamber of the at least two chambers, and at least two acquis solutions wherein a first acquis solution is contained within the top chamber, and a second acquis solution is contained within the bottom chamber. Then providing an ion-detection needle having a proximal end and a distal end, wherein an ion detector is disposed on the distal end. Followed by providing a feedback system in communication with the ion detector. Then inserting the ion-detection needle through the pierceable cap and into the first acquis solution. Acquiring a first feedback signal from the feedback system. Then inserting the ion-detection needle through the pierceable seal and into the second acquis solution. Followed by acquiring a second feedback signal from the feedback system and then comparing the differences between the first and second feedback signals.

Additionally, the method of using the point-of-use calibration system may include a first acquis solution which may have a first iron ion concentration, and a second acquis solution which may have a second iron ion concentration. Other steps may include discarding the ion-detection needle when the comparison of the first feedback signal and the second feedback signal provides no discernable difference.

The above summary is not intended to describe each and every example or every implementation of the disclosure. The description that follows more particularly exemplifies various illustrative embodiments

### BRIEF DESCRIPTION OF THE DRAWINGS

FIG. 1 is a perspective view of an embodiment of a point-of-use calibration system.

FIG. 2 is a side view of an embodiment of a point-of-use calibration system.

FIG. 3 is a cross-sectional view of the embodiment of FIG. 1 as marked.

FIG. 4 is a top view of an embodiment of a point-of-use calibration system.

FIG. 5 is a bottom view of an embodiment of a point-of-use calibration system.

FIG. 6 is a plan view of an embodiment of a point-of-use calibration system with an iron-ion detecting needle being calibrated.

### DETAILED DESCRIPTION

The present disclosure relates to iron-ion detector systems and methods used to detect iron ions in biological substances, such as bodily fluids and tissues, including blood, or non-organic equivalents, such as calibration fluids. Various embodiments of iron-ion detector systems and methods are described in detail with reference to the drawings, wherein like reference numerals may represent like parts and assemblies throughout the several views. Reference to various embodiments does not limit the scope of the biomarker detector disclosed herein. Additionally, any examples set forth in this specification are not intended to be limiting and merely set forth some of the many possible embodiments for a point-of-use calibration system for the detection of iron ions. It is understood that various omissions and substitutions of equivalents are contemplated as circumstances may suggest or render expedient. Still, these are intended to cover applications or embodiments without departing from the spirit or scope of the disclosure. Also, it is to be understood that the phraseology and terminology used herein are for the purpose of description and should not be regarded as limiting.

Surgical needles have been designed to detect specific types of fluids such as, but not limited to, blood or interstitial fluid, and to notify a user upon detection of such fluids (see U.S. Pat. No. 10,470,712 (issued 2019 Nov. 12), U.S. Pat App. 2022/0015635 (Pub. 2022-01-20), and PCT App. WO2021/133533 (Pub. 2021-07-01). To ensure such needles are effectively detecting the presence of a specific fluid prior to use, users may want to test the needle against a control. Therefore, inserting the needle into fluids having a known concentration of, for example, iron ions to see whether the needle provides an indication of iron ion presence would be useful. The present disclosure provides such a system and is described in more detail below.

More specifically, low and high ion concentrations may be prepared for the purpose of calibrating an ion detection system. In some cases, a user may desire to know when the ion detection system (for example, a needle) is in the presence of a fluid having a high ion concentration (for example, blood), but may not want the needle to indicate when it is in the presence of fluid having a low ion concentration (for example, interstitial fluid). Therefore, the disclosed calibration system can provide separate fluids having low and high ion concentrations to test against the ion detection system.

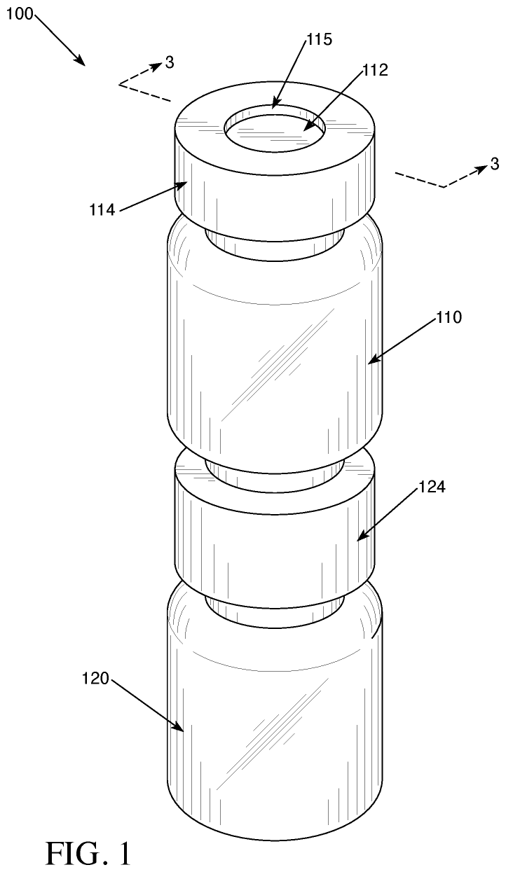

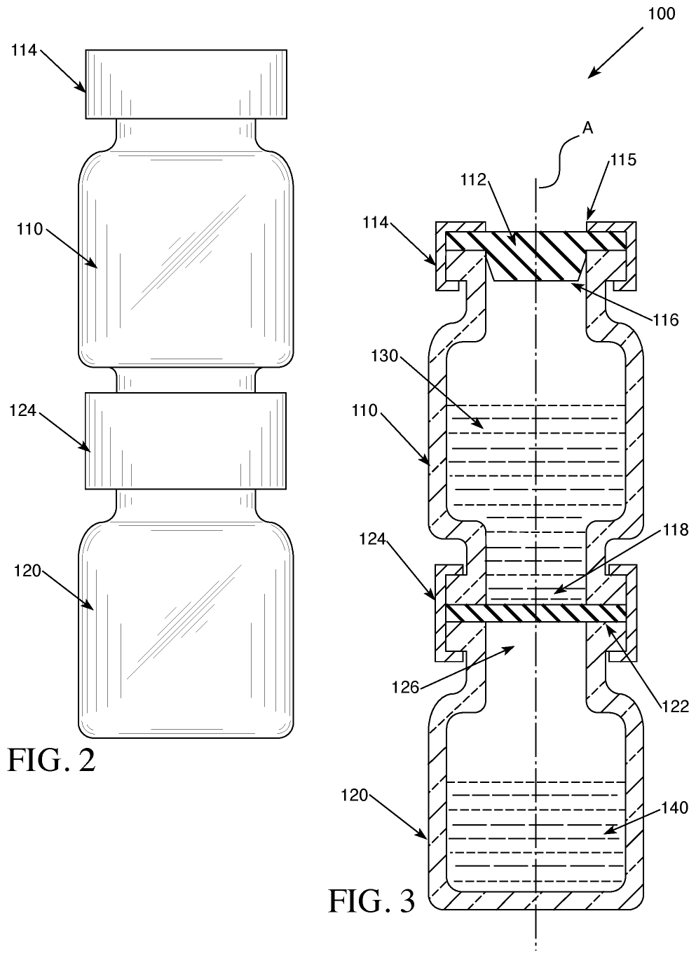

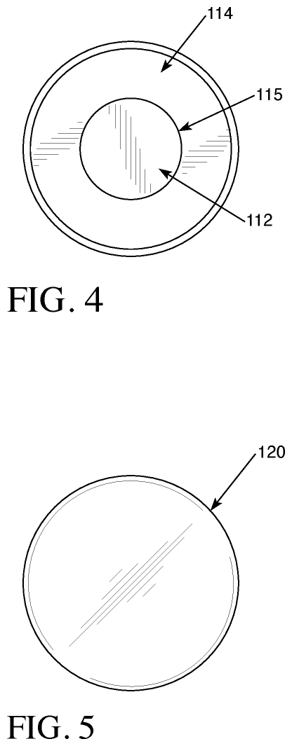

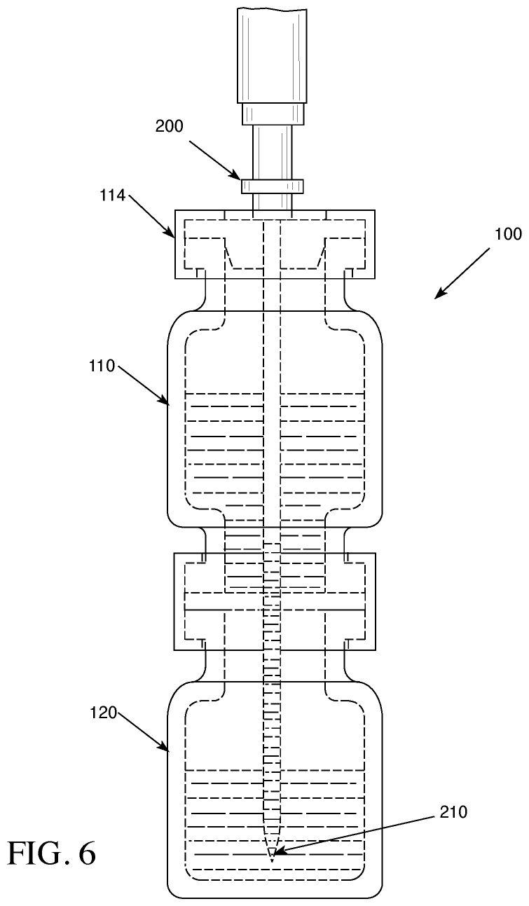

FIGS. 1-6 illustrates various examples of a point-of-use calibration system according to the present disclosure. FIG. 1 is a perspective view of the point-of-use calibration system. FIG. 2 is a side view of the point-of-use calibration system. FIG. 3 is a cross-sectional view of the point-of-use calibration system of FIG. 1. FIG. 4 is a top view of the point-of-use calibration system. FIG. 5 is a bottom view of the point-of-use calibration system. FIG. 6 is a plan view of the point-of-use calibration system with an iron-ion detecting needle being calibrated.

Preparation of the lower and higher concentrations may be achieved by creating separate acquis solutions. These differing concentrations may mimic the concentrations of ions within certain tissues and fluids within a person's body, or they may comprise iron-ion concentrations that are above and below the iron-ion concentrations typically found in blood. For example, one may wish to mimic the range of iron ion concentration that typically exists in a patient's interstitial fluid and the iron ion concentrations that typically exist in the vascular blood of a patient. Typical ranges for serum iron are: Men: 65 to 176 μg/dL, Women: 50 to 170 μg/dL, Newborns: 100 to 250 μg/dL, and Children: 50 to 120 μg/dL.

For example, a low iron ion concentration, one that is less than the typical concentration of iron ions within the vascular blood of a patient, may be created by adding a predetermined amount of iron(III) sulfate hydrate (Fe₂(SO₄)₃) that may be mixed with a predetermined amount of water to produce an acquis solution where the concentration of iron ions is less than the typical iron-ion concentration found in the vascular blood of a patient. A concentration of less than 50 μg/dL may be adequate for use as the low iron-ion calibration point as it will be less than the iron-ion concentration found in patients. To create a high iron-ion concentration, for example, one that is typical of a patient's vascular blood, a predetermined amount of iron(II) sulfate pentahydrate (FeSO₄*7H₂O) may be mixed with a predetermined amount of water to produce an acquis solution where the concentration of iron ions is similar to the iron ion concentration typical of vascular blood. For example, a concentration of 120 μg/dL may be used to cover the variance in ranges found in a patient's blood. Other iron-ion ranges are contemplated, where the ranges are based on other known biological iron-ion concentrations. For example, ranges could vary per species if an iron-ion detecting needle is intended to be used for veterinary applications. Additionally, other ranges can vary based on age or sex of the patient where an iron-ion detecting needle may be put into practice.

Acquis solutions that contain high and low ion concentrations may be used to calibrate an ion detection system. Specifically, such ion detection systems that may reside within the end of a needle, as mentioned above. For example, in FIG. 3 a multi-chambered vial 100 may include a low iron ion solution (as described supra) 130 that can be disposed within a first chamber 110 and a high iron ion solution (as described supra) 140 that can be disposed within a second chamber 120. The first chamber 110 and the second chamber 120 may be prevented from mixing with the use of a rubber seal 122 being disposed between the two chambers, wherein each chamber includes an opening opposite the other where the rubber seal 122 resides. The first chamber 110 may include a lower opening 118 where the rubber seal 122 is disposed, while the second chamber 120 may include an opening 126 where the opposite side of the rubber seal 122 may be disposed. The combined structure of the first chamber 110, the second chamber 120, and the rubber seal 122 may be secured with the use of a securing band 124. The securing band 124 may consist of aluminum or other such metals typically used in the art of vial construction and may enclose and secure the lower opening 118 and the opening 126. The multi-chambered vial 100 may be composed of sterile depyrogenated glass; for example, SHOTT FIIOLAX glass. The point-of-use calibration system can be used to calibrate needles with proximal and distal ends that may contain an ion detector in their distal end. In such needles with an ion detector, the distal end may be inserted into the first chamber 110 to obtain a first detection, and then into the second chamber 120 to obtain a second detection.

The first chamber 110 may include an additional opening opposite the lower opening 118, this being the first opening 116. An additional rubber cap 112 may be disposed within the first opening 116. The first chamber 110 may also include a securing band 114 that may consist of aluminum or other such metals typically used in the art of vial construction and may enclose the rubber seal 122 and first opening 116. The securing band 114 may include an access point 115 that provides an opening within the securing band 114; this access point 115 may provide a needle access to the first chamber 110 and the second chamber 120 when the needle is inserted through the rubber cap 112 and seal 122.

In some embodiments, the first chamber 110 is a different shape than the second chamber 120. More specifically, the first chamber 110 may be structured and configured such that it has an upper and a lower neck, whereas the second chamber 120 may be structured and configured such that it only has an upper neck. The upper and lower necks of the first chamber 110 may be narrower than the main body of the chamber in which the majority of the solution 130 resides. Having such a shape enables the first chamber 110 to be more easily paired to the second chamber 120 since the lower neck of the first chamber 110 can be approximately the same size as (for example, the same interior and exterior circumferences), and therefore aligned with, the neck of the second chamber 120. The openings may correspond to the locations of the necks. For example, the first opening 116 can correspond to the upper neck of the first chamber 110, the lower opening can correspond to the lower neck of the first chamber 110, and the opening 126 can correspond to the upper neck of the second chamber 120. However, the two chambers, 110 and 120, do not have to have a different construction.

It is contemplated that some embodiments of a multi-chambered vial may include identical first and second chambers. In such embodiments, both chambers may have upper and lower necks and upper and lower openings, wherein the upper opening in one chamber includes a rubber cap, and the lower opening in a second chamber may also include a cap. The two chambers may then be aligned on a common axis and have a rubber seal disposed between the two chambers at their remaining openings. Such an embodiment may reduce the cost of manufacturing as the two chambers could potentially be constructed with multiples of the same structure.

The rubber seal 122 and rubber cap 112 can be structured and configured to allow the passage of an ion detection system needle by being a pierceable rubber. Such pierceable rubber, e.g., TEFLON-coated non-latex chorobutyrl rubber, may allow the needle to pass through the seals without allowing the mixing of the two acquis solutions, i.e., the low iron ion solution 130 and the high iron ion solution 140.

FIG. 6 illustrates an embodiment of an ion-detection needle 200 being calibrated within the multi-chambered vial 100, where the first chamber 110 may contain a first iron-ion concentration and where the second chamber 120 may contain a second iron-ion concentration. The ion-detection needle 200 may also include an ion detector 210 within the distal end of the ion-detection needle 200. The proximal end of the ion-detection needle 200 may be connected to a feedback system or a syringe, which may include a connection therethrough to a feedback system (not illustrated). One possible way to achieve the passage of an ion detection system needle 200 into both the first chamber 110 and the second chamber 120 may be to align the first chamber 110, second chamber 120, the rubber seal 122, and the rubber cap 112 with single common axis A, as illustrated in FIG. 3. Such a common axis will provide a cylinder-like structure for the multi-chambered vial 100. In addition, the ion detection system may be configured in such a way that feedback is provided to an end-user that may alert them to the presence of different ion concentrations in real-time. For example, an ion detector 210 may be located in the ion detection needle 200 that is introduced to an ion-containing solution and may provide a signal to a processor in a feedback system (not illustrated) which may, in turn, provide feedback to a user. More specifically, the feedback system can be comprised of a readout on a connected screen with an ion concentration, an audible tone that varies with the level of ions present in a solution, an emitted light that varies in intensity or color with the level of ions present in a solution, an indicator light that can provide a binary, on or off result, and combinations thereof. Alternatively, the ion detection system may operate on a threshold basis such that it may not trigger an alert or feedback unless it detects a minimum amount of ions in the solution. For example, a light indicator may either be on or off depending on whether the predetermined ion threshold has been detected (i.e., the light is on if the threshold has been reached and off if it has not). As such, the first chamber 110 may include the low iron-ion solution 130 while the second chamber 120 may include the high iron-ion solution 140, with the goal being that the ion-detection system would not trigger an alert for the user when it is inserted into the low iron-ion solution 130, but it would trigger an alert for the user when it is inserted into the high iron-ion solution 140. The solutions may also be presented in the opposite chambers (i.e., the high iron-ion solution 140 in the first chamber 110 and the low iron-ion solution 130 in the second chamber 120) to see whether the ion-detection system triggers in the first chamber 110 and then goes silent in the second chamber 120.

With an aligned set of chambers along a common axis, a point-of-use calibration system can mimic the use of an ion-detection system needle with a patient. The insertion of the ion-detection system needle can first detect a low iron ion concentration in the first chamber, similar to the insertion into the interstitial tissue of a patient. The system may further mimic the use of an ion-detection system needle with a patient when the needle is inserted further into the second chamber containing a higher iron-ion concentration, similar to the insertion into a patient's vascular blood. In a real-world scenario, assuming the user does not want to deliver the medicine or fluids directly into the bloodstream, the user may then want to pull the needle back into the interstitial tissue and be alerted (either by a readout or by lack of a signal) once they are in the lower iron-ion concentration. The ion-detection system can then indicate to the user that they are no longer in the bloodstream and that it is appropriate to deliver the corresponding medicine or fluid.

Other embodiments for the point-of-use calibration system for an ion-detection system may include ion-containing chambers within separate vials. For example, a first vial with a first chamber may contain a low-ion concentration, and a second vial with a second chamber may contain a high-ion concentration. Further embodiments may include a point-of-use calibration system that can include multiple chambers where there are three or more chambers aligned on a common axis. In such embodiments, chambers would include upper and lower openings similar to the first chamber 110 in FIG. 1. Each chamber may be stacked upon each other and may be separated by a seal leaving the lowest chamber with only one opening, similar to the second chamber 120. The systems where there are more than two chambers may need to reduce their overall height in proportion to the two-chamber systems in order to accommodate various needle lengths.

Persons of ordinary skill in arts relevant to this disclosure and subject matter hereof will recognize that embodiments may comprise fewer features than illustrated in any individual embodiment described by example or otherwise contemplated herein. Embodiments described herein are not meant to be an exhaustive presentation of ways in which various features may be combined and/or arranged. Accordingly, the embodiments are not mutually exclusive combinations of features; rather, embodiments can comprise a combination of different individual features selected from different individual embodiments, as understood by persons of ordinary skill in the relevant arts. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless stated that a specific combination is not intended. Furthermore, it is also intended to include features of a claim in any other independent claim, even if this claim is not directly made dependent on the independent claim.

1. A point-of-use calibration system for an ion detection system comprising:
at least a first chamber and a second chamber,
wherein the first chamber includes a first ionic solution, and
wherein the second chamber includes a second ionic solution.

2. The point-of-use calibration system for an ion detection system of claim 1, further comprising:
the first chamber having an upper opening and a lower opening;
the second chamber having an opening;
a seal disposed between the lower opening of the first chamber and the opening of the second chamber; and
a cap disposed on the upper opening of the first chamber,
wherein the first chamber, the second chamber, the seal, and the cap are aligned on a common axis.

3. The point-of-use calibration system of claim 2, wherein the first ionic solution contains a first concentration of iron ions, and the second ionic solution contains a second concentration of iron ions.

4. The point-of-use calibration system of claim 3, wherein the seal and the cap are constructed of a needle-pierceable material.

5. The point-of-use calibration system of claim 4, further comprising an ion-detection needle having a proximal end and a distal end, wherein an ion detector is disposed on the distal end.

6. The point-of-use calibration system of claim 5, wherein the proximal end of the ion-detection needle is disposed onto a syringe, and the ion detector is in communication with a feedback system.

7. The point-of-use calibration system of claim 6, wherein the feedback system is selected from the group consisting of an indicator light, a numeric digital display, a variable tone generator, or a variable color display, and combinations thereof.

8. A multi-chambered vial comprising:
at least two chambers wherein the at least two chambers are aligned on a common vertical axis;
a first chamber of the at least two chambers is a top chamber and a second chamber of the at least two chambers is a bottom chamber, wherein the top chamber has an upper opening and a lower opening, and the lower chamber has an opening;
a pierceable cap disposed on a top opening of the top chamber;
a pierceable seal disposed between every chamber of the at least two chambers; and
at least two acquis solutions wherein the at least two acquis solutions consist of different ionic concentrations.

9. The multi-chambered vial of claim 8, wherein a retaining ring is disposed on the pierceable cap and around each pierceable seal.

10. The multi-chambered vial of claim 9, wherein the at least two chambers include a third chamber having an upper opening and a lower opening, wherein the third chamber is disposed between the top chamber and the bottom chamber, and wherein the third chamber has a different ionic concentration than the first and second chambers.

11. A method for using a point-of-use calibration system for an ion detection system comprising the steps of:
providing a multi-chambered vial comprising:
at least two chambers wherein the at least two chambers are aligned on a common vertical axis;
a first chamber of the at least two chambers is a top chamber and a second chamber of the at least two chambers is a bottom chamber, wherein the top chamber has an upper opening and a lower opening, and the lower chamber has an opening;
a pierceable cap disposed on a top opening of the top chamber;
a pierceable seal disposed between every chamber of the at least two chambers; and
at least two acquis solutions wherein a first acquis solution is contained within the top chamber, and a second acquis solution is contained within the bottom chamber;
providing an ion-detection needle having a proximal end and a distal end, wherein an ion detector is disposed on the distal end;
providing a feedback system that is in communication with the ion detector;
inserting the ion-detection needle through the pierceable cap and into the first acquis solution;
acquiring a first feedback signal from the feedback system;
inserting the ion-detection needle through the pierceable seal and into the second acquis solution;
acquiring a second feedback signal from the feedback system;
comparing the differences between the first feedback signal and the second feedback signal.

12. The method of claim 11, wherein the first acquis solution has a first iron ion concentration, and the second acquis solution has a second iron ion concentration.

13. The method of claim 11, further comprising the step of discarding the ion-detection needle when the comparison of the first feedback signal and the second feedback signal provides no discernable difference.
